(19) United States
(12) Patent Application Publication
(10) Pub. No.: US 2026/0097209 A1
(43) Pub. Date: Apr. 9, 2026

(54) IDENTIFYING ARTIFACTS FROM SECONDARY IMPLANTED DEVICES DURING CLOSED-LOOP STIMULATION

(71) Applicant: Medtronic, Inc., Minneapolis, MN (US)

(72) Inventors: Malgorzata Maria Straka, Blaine, MN (US); Joshua J. Nedrud, Roseville, MN (US); Joel A. Anderson, Brooklyn Park, MN (US); Kristin N. Hageman, Dayton, MN (US); Leonid M. Litvak, Bet Shemesh (IL); Annemarie K. Brinda, Columbia Heights, MN (US); Philip E. Tracton, Porter Ranch, CA (US); Thaddeus S. Brink, St. Paul, MN (US)

(21) Appl. No.: 19/343,846

(22) Filed: Sep. 29, 2025

Related U.S. Application Data
(60) Provisional application No. 63/703,745, filed on Oct. 4, 2024.

(51) Int. Cl. A61N 1/36

(52) U.S. Cl. CPC A61N 1/36125; A61N 1/36062; A61N 1/36139

(57) ABSTRACT

A medical device includes processing circuitry configured to: receive information for a sensed evoked signal that is evoked due to stimulation delivery; determine whether noise generated from delivery of stimulation by a secondary medical device is present in the sensed evoked signal; and process the sensed evoked signal based on the determination of whether the noise generated from delivery of stimulation by the secondary medical device is present in the sensed evoked signal.

### CROSS-REFERENCE TO RELATED APPLICATION

This application claims the benefit of and priority to U.S. Provisional Ser. No. 63/703,745 filed Oct. 4, 2024, the entire disclosure of which is incorporated by reference herein.

### TECHNICAL FIELD

This disclosure generally relates to controlling stimulation based on sensed signals.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

Electrical stimulation may be delivered to a patient by the medical device in a train of electrical pulses, and parameters of the electrical pulses may include a frequency, an amplitude, a pulse width, and a pulse shape. An evoked compound action potential (ECAP) is synchronous firing of a population of neurons which occurs in response to the application of a stimulus including, in some cases, an electrical stimulus by a medical device. The ECAP may be detectable as being a separate event from the stimulus itself, and the ECAP may reveal characteristics of the effect of the stimulus on the nerve fibers.

### SUMMARY

In general, systems, devices, and techniques are described for determining whether noise generated from delivery of stimulation by a secondary medical device is present in a sensed evoked signal of a primary medical device. For closed-loop therapy, the primary medical device may receive information for a sensed evoked signal that is sensed within a time window that is relative to a time when stimulation is delivered that evokes the evoked signal. The primary medical device may use the sensed evoked signal for various purposes, including closed-loop therapy. That is, the primary medical device may increase or decrease stimulation based on the sensed evoked signal.

In addition to content of the evoked signal, the sensed evoked signal may include noise. There may be various noise sources, one of which may be stimulation signals delivered by a secondary medical device. The noise forms artifacts in the sensed evoked signal, and can impact the closed-loop therapy (e.g., decrease stimulation due to the artifact, and not because actual content of the evoked signal).

In accordance with one or more examples, processing circuitry (e.g., of the primary medical device or some other medical device, such as programmer or server) may determine whether the sensed evoked signal includes noise generated from delivery of stimulation by a secondary medical device. The processing circuitry may process the sensed evoked signal based on a determination of whether the noise generated from delivery of stimulation by the secondary medical device is present in the sensed evoked signal.

In one example, this disclosure describes a medical device comprising: processing circuitry configured to: receive information for a sensed evoked signal that is evoked due to stimulation delivery; determine whether noise generated from delivery of stimulation by a secondary medical device is present in the sensed evoked signal; and process the sensed evoked signal based on the determination of whether the noise generated from delivery of stimulation by the secondary medical device is present in the sensed evoked signal.

In one example, the disclosure describes a method for noise detection, the method comprising: receiving information for a sensed evoked signal that is evoked due to stimulation delivery; determining whether noise generated from delivery of stimulation by a secondary medical device is present in the sensed evoked signal; and processing the sensed evoked signal based on the determination of whether the noise generated from delivery of stimulation by the secondary medical device is present in the sensed evoked signal.

In one example, the disclosure describes a computer-readable storage medium storing instructions thereon that when executed cause one or more processors to receive information for a sensed evoked signal that is evoked due to stimulation delivery; determine whether noise generated from delivery of stimulation by a secondary medical device is present in the sensed evoked signal; and process the sensed evoked signal based on the determination of whether the noise generated from delivery of stimulation by the secondary medical device is present in the sensed evoked signal.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) configured to deliver spinal cord stimulation (SCS) therapy, a cardiac device, and an external programmer, in accordance with one or more techniques of this disclosure.

FIG. 2 is a block diagram illustrating an example configuration of components of an IMD, in accordance with one or more techniques of this disclosure.

FIG. 3 is a block diagram illustrating an example configuration of components of an example external programmer, in accordance with one or more techniques of this disclosure.

FIG. 4A is a graph of example of a sensed evoked signal that is sensed within a time window that is relative to a time when stimulation is delivered that evokes the evoked signal that is sensed.

FIG. 4B is a graph of the actual evoked signal.

FIG. 4C is a graph of an example template used for determining whether noise generated from delivery of stimulation by a secondary medical device is present in the sensed evoked signal.

FIG. 5 is a graph of an example of a plurality of amplitude spikes in one or more instances of previously sensed evoked signals for determining whether the noise generated from delivery of stimulation by the secondary medical device is present in the sensed evoked signal.

FIG. 6A is a graph of example of a first sensed evoked signal that is sensed with a first sensing channel.

FIG. 6B is a graph of example of a second sensed evoked signal that is sensed with a second sensing channel.

FIG. 7 is a flow chart illustrating an example method of operation, in accordance with one or more techniques of this disclosure.

### DETAILED DESCRIPTION

The disclosure describes examples of medical devices, systems, and techniques for determining whether noise generated from delivery of stimulation by a secondary medical device is present in a sensed evoked signal. For example, processing circuitry may receive information for a sensed evoked signal that is evoked due to stimulation delivery. For instance, the processing circuitry may receive information for a sensed evoked signal that is sensed within a time window that is relative to a time when stimulation is delivered that evokes the evoked signal that is sensed. One example of the sensed evoked signal is a sensed evoked compound action potential (ECAP) signal. The sensed evoked signal includes content of the ECAP, and possibly includes noise.

Electrical stimulation therapy is typically delivered to a target tissue (e.g., nerves of the spinal cord or muscle) of a patient via two or more electrodes. Parameters of the electrical stimulation therapy (e.g., electrode combination, voltage or current amplitude, pulse width, pulse frequency, etc.) are selected by a clinician and/or the patient to provide relief from various symptoms, such as pain, nervous system disorders, muscle disorders, etc. Various thresholds, such as a perception threshold and/or discomfort threshold may be determined for the patient and used to select and/or recommend parameters of the stimulation therapy.

For ease of description, the examples are described with respect to ECAPs, but the techniques are applicable to other evoked signals. ECAPs are a measure of neural recruitment because each ECAP signal represents the superposition of electrical potentials generated from a population of axons firing in response to an electrical stimulus (e.g., a stimulation pulse). Changes in a characteristic (e.g., an amplitude of a portion of the signal or area under the curve of the signal) of an ECAP signals occur as a function of how many axons have been activated by the delivered stimulation pulse. For a given set of parameter values that define the stimulation pulse and a given distance between the electrodes and target nerve, the detected ECAP signal may have a certain characteristic value (e.g., amplitude).

In some examples, effective stimulation therapy may rely on a certain level of neural recruitment at a target nerve. This effective stimulation therapy may provide relief from one or more conditions (e.g., patient perceived pain) without an unacceptable level of side effects (e.g., overwhelming perception of stimulation).

Although the system may adjust one or more stimulation parameters according to the one or more characteristics of the sensed ECAP signal, for example, to compensate for the change in distance between electrodes and nerves, the precision of such adjustments is dependent on accurately determining the characteristics of the ECAP signal. For example, noise such as stimulation artifacts and/or linear or exponential background noise may interfere with accurate determinations of the magnitude of one or more peaks within the ECAP signal. Stimulation artifacts typically have amplitudes many times that of the ECAP signal and can at least partially overlap with the ECAPs from nerves. Inaccurate ECAP characterization can reduce the effectiveness of using ECAP characteristic values for automatically adjusting stimulation parameters and result in less efficacious therapy for the patient. Moreover, manually identifying patient thresholds, such as a perception threshold, can be time consuming and rely on subjective feedback from the patient. Therefore, clinicians may be pressed for time when setting up stimulation, perception thresholds may be inaccurate, and patients may need to return to the clinic in order to update the stimulator programming for example. These issues may reduce the likelihood that the patient receives efficacious therapy that could be provided.

Using sensed evoked signals, like ECAPs, to control therapy is referred to as closed-loop therapy, closed-loop stimulation, or closed-loop neuromodulation. As described, closed-loop devices such as spinal cord stimulation (SCS) devices may detect spinal ECAPs to control the therapeutic stimulation amplitudes delivered to patients. In addition to sensing the targeted ECAPs, other biosignals may also be acquired such as far-field cardiac activity or muscle activity by the primary sensing device. Moreover, if the patient has an additional active implanted device such as a pacemaker or a combination of more than one SCS device, deep brain stimulation (DBS) device, or pelvic health devices, the electrical impulses from the “secondary device” may be detected by the sensing device (e.g., primary device). This may act as a noise or artifact that obscures the ECAP in the primary device, thus impacting the accuracy in the closed loop algorithm of the primary device.

Stated another way, a primary medical device may be configured to sense evoked signals for use in closed-loop stimulation. However, if sensing of the evoked signals coincides with a secondary medical device delivering stimulation (e.g., a pacemaker delivering a pacing signal, etc.), the primary medical device senses the stimulation delivered by the secondary medical device in combination with the evoked signals. That is, the sensed evoked signals includes content of the actual evoked signals and content from the stimulation by the secondary medical device.

If the primary medical device uses the sensed evoked signals for controlling stimulation, the primary medical device may adjust the stimulation parameters in a less optimal manner because the noise generated from delivery of the stimulation by the secondary medical device obscures the content of the evoked signal. With the techniques described in this disclosure, the processing circuitry may be configured to determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal. In this manner, the operation of closed-loop stimulation may be improved, especially for patients with multiple implanted medical devices.

For instance, it may be possible to remove closed-loop stimulation for patients with multiple implanted medical devices. While such an option may be satisfactory in some cases, having the benefits of closed-loop stimulation may be unavailable. For example, some patients with pacemakers may not be able to benefit from the closed-loop feature.

With the example techniques, the processing circuitry may determine whether noise generated from delivery of stimulation by a secondary medical device is present in the sensed evoked signal, and process the sensed evoked signal accordingly. The ability to correctly identify and distinguish the noise generated from delivery of stimulation by a second medical device may enable better therapies to be provided to the patient.

There may be various techniques in which the processing circuitry may determine whether noise generated from delivery of stimulation by a secondary medical device is present in the sensed evoked signal. The various techniques may function separately or in combination. In general, the processing circuitry (e.g., of the primary medical device) may use a neural network or other algorithm to identify artifacts (e.g., noise) from additional electrical stimulation devices and remove/reduce their impact in the closed-loop control from the evoked signals (e.g., ECAPs).

For example, for a neural network (e.g., machine learning (ML)/artificial intelligence (AI)) technique, the processing circuitry may execute a trained model. The training data used to train the trained model may be based on sensed evoked signals where stimulation from another device is turned on during the time window when the evoked signals are sensed. For instance, the training data may include sensed evoked signals where stimulation from another device is turned off, and sensed evoked signals where stimulation from another device is turned on. A medical professional may evaluate the sensed evoked signals where stimulation from another device is turned on to identify the noise (e.g., artifacts). This training data may then be used to train the trained model, and form as the ground truth. The trained model may then be available for execution for determining whether noise generated from delivery of stimulation by a secondary medical device is present in the sensed evoked signal. In addition, one of the outputs from execution of the trained model may be locations of the noise (e.g., how the noise is manifesting in the sensed evoked signal) and/or ways to remove that noise (e.g., subtract out the noise). Accordingly, the trained model may be configured to process the sensed evoked signal based on a determination of whether the noise generated from delivery of stimulation by the secondary medical device is present in the sensed evoked signal, such as output the sensed evoked signal with the noise removed. Additionally, the training model may be configure to use the continuous recording mode (e.g., continuous sensing and not only after stimulation) to use for training.

The following are some additional examples of techniques that the processing circuitry may use for determining whether noise generated from delivery of stimulation by a secondary medical device is present in the sensed evoked signal, and processing the sensed evoked signal based on a determination of whether the noise generated from delivery of stimulation by the secondary medical device is present in the sensed evoked signal. The techniques may be used together or separately, and together or separately with the neural network techniques.

As one example, the processing circuitry may use template matching techniques. As another example, noise generated from delivery of stimulation by a secondary medical device may be periodic, and appears as spikes in the sensed evoked signal. The processing circuitry may determine the period of the noise, and use that to estimate when noise would be present in a current sensed evoked signal. If noise is present (e.g., in form of a spike), the processing circuitry may determine that noise generate from delivery of stimulation from a secondary medical device is present.

As further examples, the secondary medical device may be relatively distant from the primary medical device, in some cases. For such cases, the noise generated by the delivery of stimulation from the secondary medical device would appear on multiple sensing channels (e.g., multiple different electrodes used for sensing). The processing circuitry may determine whether there is correlation between the signals sensed using different sensing channels. If there is correlation, the processing circuitry may determine that there is noise generated by the delivery of stimulation from the secondary medical device.

As another example, the sensing of the evoked signal may occur within a time window that is relative to a time when stimulation is delivered that evokes the evoked signal that is sensed. However, the primary medical device may be configured to sense signals in a continuous mode. Since the continuous mode sensing is sensing more often than the sensing of the evoked signal, the processing circuitry may determine whether there is noise in the sensed signal that is sensed in the continuous mode. The processing circuitry may determine whether the noise generated from delivery of stimulation by the secondary medical device is present in the sensed evoked signal based on the determination of whether noise generated from delivery of the stimulation by the secondary medical device is present in the sensed signal that is sensed in the continuous mode.

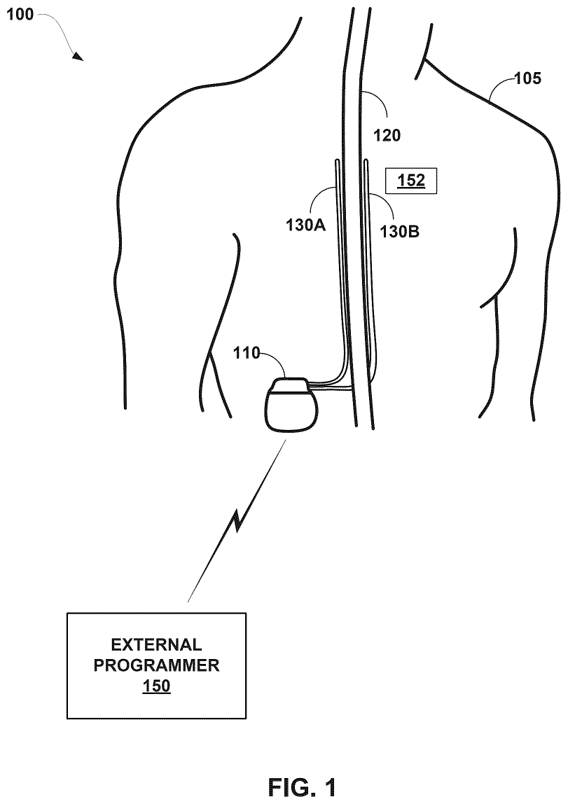

FIG. 1 is a conceptual diagram illustrating an example system 100 that includes an implantable medical device (IMD) 110 configured to deliver SCS therapy, cardiac device 152, and an external programmer 150, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As illustrated, patient 105 is implanted with IMD 110 and cardiac device 152. IMD 110 may be considered as a primary medical device, and cardiac device 152 may be considered as a secondary medical device. For ease of description only, the secondary medical device is illustrated as cardiac device 152. One example of cardiac device 152 is a pacemaker. However, the techniques are not so limited. The secondary medical device may be another SCS device, a DBS device, a device for pelvic therapy, or another device that delivers stimulation.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes of electrodes of leads 130A and/or 130B (collectively, “leads 130”), e.g., for relief of chronic pain or other symptoms. In other examples, IMD 110 may be coupled to a single lead carrying multiple electrodes or more than two leads each carrying multiple electrodes. In some examples, the stimulation signals, or pulses, may be configured to elicit detectable evoked signals (e.g., ECAP) signals that IMD 110 may use to determine the posture state occupied by patient 105 and/or determine how to adjust one or more parameters that define stimulation therapy. IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless.

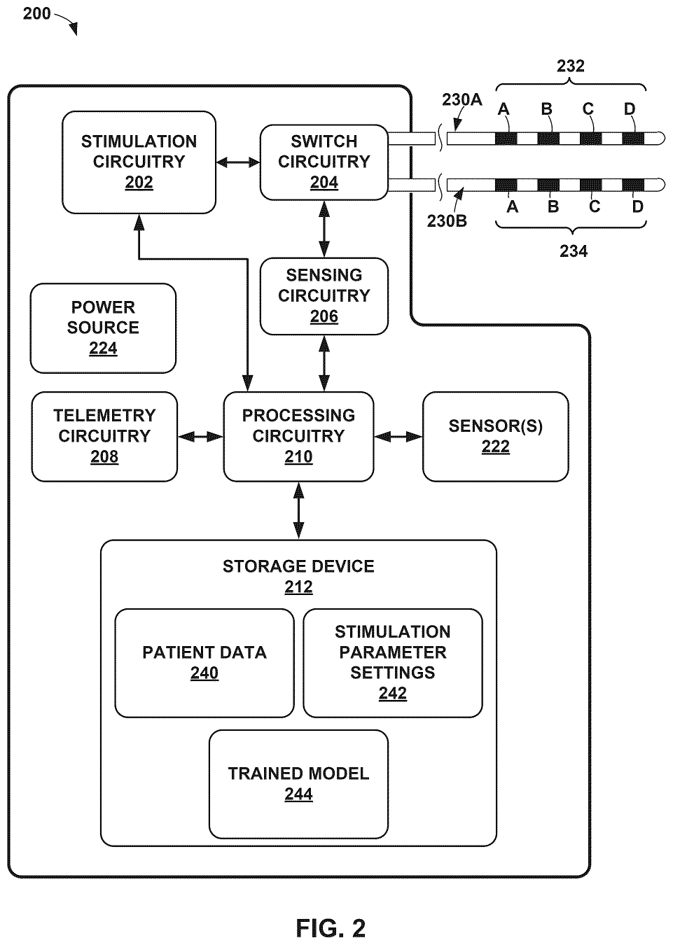

IMD 110 may be constructed of any polymer, metal, or composite material sufficient to house the components of IMD 110 (e.g., components illustrated in FIG. 2) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

Electrical stimulation energy, which may be constant current or constant voltage-based pulses, for example, is delivered from IMD 110 to one or more target tissue sites of patient 105 via one or more electrodes (not shown) of implantable leads 130. In the example of FIG. 1, leads 130 carry electrodes that are placed adjacent to the target tissue of spinal cord 120. One or more of the electrodes may be disposed at a distal tip of a lead 130 and/or at other positions at intermediate points along the lead. Leads 130 may be implanted and coupled to IMD 110. The electrodes may transfer electrical stimulation generated by an electrical stimulation generator in IMD 110 to tissue of patient 105. Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The electrodes of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having eight ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 110 through the electrodes of leads 130 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 100 based on one or more factors or user input.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of DBS, peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 105.

In some examples, lead 130 includes one or more sensors configured to allow IMD 110 to monitor one or more parameters of patient 105, such as patient activity, pressure, temperature, or other characteristics. The one or more sensors may be provided in addition to, or in place of, therapy delivery by lead 130.

IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

IMD 110 is configured to generate and deliver electrical stimulation therapy to a target stimulation site within patient 105 via the electrodes of leads 130 to patient 105 according to one or more therapy stimulation programs. A therapy stimulation program defines values for one or more parameters (e.g., a parameter set) that define an aspect of the therapy delivered by IMD 110 according to that program. For example, a therapy stimulation program that controls delivery of stimulation by IMD 110 in the form of pulses may define values for voltage or current pulse amplitude, pulse width, pulse rate (e.g., pulse frequency), electrode combination, pulse shape, etc. for stimulation pulses delivered by IMD 110 according to that program.

Furthermore, IMD 110 may be configured to deliver stimulation to patient 105 via a combination of electrodes of leads 130, alone or in combination with an electrode carried by or defined by an outer housing of IMD 110 in order to detect ECAP signals. The tissue targeted by the stimulation may be the same or similar tissue targeted by the electrical stimulation therapy, but IMD 110 may deliver stimulation pulses for ECAP signal detection via the same, at least some of the same, or different electrodes.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from external programmer 150 to control stimulation, such as electrical stimulation therapy to develop the growth curve. For example, external programmer 150 may transmit therapy stimulation programs, stimulation parameter adjustments, therapy stimulation program selections, user input, or other information to control the operation of IMD 110, e.g., by wireless telemetry or wired connection.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy, when a patient perceives stimulation being delivered or when a patient terminates due to comfort level. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

As described herein, information may be transmitted between external programmer 150 and IMD 110. Therefore, IMD 110 and external programmer 150 may communicate via wireless communication using any techniques known in the art. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

In some examples, IMD 110, in response to commands from external programmer 150, delivers electrical stimulation therapy according to a plurality of therapy stimulation programs to a target tissue site of the spinal cord 120 of patient 105 via electrodes (not depicted) on leads 130. In some examples, IMD 110 modifies therapy stimulation programs as therapy needs of patient 105 evolve over time. For example, the modification of the therapy stimulation programs may cause the adjustment of at least one parameter of the plurality of therapy pulses. When patient 105 receives the same therapy for an extended period, the efficacy of the therapy may be reduced. In some cases, parameters of the plurality of therapy pulses may be automatically updated. In some examples, IMD 110 may detect ECAP signals from pulses delivered for the purpose of providing therapy to the patient.

In some examples, efficacy of electrical stimulation therapy may be indicated by one or more characteristics of an action potential that is evoked by a stimulation pulse delivered by IMD 110, for example by determining an estimated neural response using the characteristic value of the ECAP signal. Electrical stimulation therapy delivery by leads 130 of IMD 110 may cause neurons within the target tissue to evoke a compound action potential that travels up and down the target tissue, eventually arriving at sensing electrodes of IMD 110. Furthermore, stimulation pulses may also elicit at least one ECAP signal, and ECAPs responsive to stimulation may also be a surrogate for the effectiveness of the therapy and/or the intensity perceived by the patient. The amount of action potentials (e.g., number of neurons propagating action potential signals) that are evoked may be based on the various parameters of electrical stimulation pulses such as amplitude, pulse width, frequency, pulse shape (e.g., slew rate at the beginning and/or end of the pulse), etc. The slew rate may define the rate of change of the voltage and/or current amplitude of the pulse at the beginning and/or end of each pulse or each phase within the pulse. For example, a very high slew rate indicates a steep or even near vertical edge of the pulse, and a low slew rate indicates a longer ramp up (or ramp down) in the amplitude of the pulse. In some examples, these parameters contribute to an intensity of the electrical stimulation. In addition, a characteristic of the ECAP signal (e.g., an amplitude) may change based on the distance between the stimulation electrodes and the nerves subject to the electrical field produced by the delivered control stimulation pulses.

Example techniques for adjusting stimulation parameter values for pulses (e.g., pulses configured to contribute to therapy for the patient) are based on comparing the value of a characteristic of a measured ECAP signal to a target ECAP characteristic value. In some examples, the target ECAP characteristic value may be the estimated neural threshold or a value calculated based on the estimated neural threshold (e.g., a percentage below or above 100% of the estimated neural threshold). During delivery of control stimulation pulses defined by one or more ECAP test stimulation programs, IMD 110, via two or more electrodes interposed on leads 130, senses electrical potentials of tissue of the spinal cord 120 of patient 105 to measure the electrical activity of the tissue. IMD 110 senses ECAPs from the target tissue of patient 105, e.g., with electrodes on one or more leads 130 and associated sense circuitry. In some examples, IMD 110 receives a signal indicative of the ECAP from one or more sensors, e.g., one or more electrodes and circuitry, internal or external to patient 105. Such an example signal may include a signal indicating an ECAP of the tissue of patient 105.

In the example of FIG. 1, IMD 110 is described as performing a plurality of processing and computing functions. However, external programmer 150 instead may perform one, several, or all of these functions. In this alternative example, IMD 110 functions to relay sensed signals to external programmer 150 for analysis, and external programmer 150 transmits instructions to IMD 110 to adjust the one or more parameters defining the electrical stimulation therapy based on analysis of the sensed signals. For example, IMD 110 may relay the sensed signal indicative of an ECAP to external programmer 150. External programmer 150 may compare the parameter value of the ECAP to the target ECAP characteristic value relative to an estimated neural response, and in response to the comparison, external programmer 150 may instruct IMD 110 to adjust one or more stimulation parameter that defines the electrical stimulation pulses delivered to patient 105.

In the example techniques described in this disclosure, the stimulation parameters and the target ECAP characteristic values associated with the estimated neural response may be initially set at the clinic but may be set and/or adjusted at home by patient 105. For example, the target ECAP characteristics may be changed to match, be a fraction of, or a multiplier of, a stimulation threshold. In some examples, target ECAP characteristics may be specific to respective different posture states of the patient. Once the target ECAP characteristic values are set, the example techniques allow for automatic adjustment of parameter values that define stimulation pulses to maintain consistent volume of neural activation and consistent perception of therapy for the patient. The ability to change the stimulation parameter values may also allow the therapy to have long term efficacy, with the ability to keep the intensity of the stimulation (e.g., as indicated by the ECAP) consistent by comparing the measured ECAP values to the target ECAP characteristic value. In addition, or alternatively, to maintaining stimulation intensity, IMD 110 may monitor the characteristic values of the ECAP signals to limit one or more parameter values that define stimulation pulses. IMD 110 may perform these changes without intervention by a physician or patient 105.

In some examples, the system changes the target ECAP characteristic value over a period of time, such as according to a change to a stimulation threshold (e.g., a perception threshold or detection threshold). The system may be programmed to change the target ECAP characteristic in order to adjust the intensity of stimulation pulses to provide varying sensations to the patient (e.g., increase or decrease the volume of neural activation). Although the system may change the target ECAP characteristic value, received ECAP signals may still be used by the system to adjust one or more parameter values of the stimulation pulse in order to meet the target ECAP characteristic value.

One or more devices within system 100, such as IMD 110 and/or external programmer 150, may perform various functions as described herein. For example, IMD 110 may include stimulation circuitry configured to deliver electrical stimulation, sensing circuitry configured to sense a plurality ECAP signals, and processing circuitry. The processing circuitry may be configured to control the stimulation circuitry to deliver a plurality of electrical stimulation pulses having different amplitude values and control the sensing circuitry to detect, after delivery of each electrical stimulation pulse of the plurality of electrical stimulation pulses, a respective ECAP signal of the plurality of ECAP signals, and to determine ECAP characteristic values for each of the ECAP signals.

In some examples, IMD 110 may include the stimulation circuitry, the sensing circuitry, and the processing circuitry. However, in other examples, one or more additional devices may be part of the system that performs the functions described herein. For example, IMD 110 may include the stimulation circuitry and the sensing circuitry, but external programmer 150 or other external device may include the processing circuitry that at least determines the estimated neural threshold of the patient. IMD 110 may transmit the sensed ECAP signals, or data representing the ECAP signal, to external programmer 150, for example. Therefore, the processes described herein may be performed by multiple devices in a distributed system. In some examples, system 100 may include one or more electrodes that deliver and/or sense electrical signals. Such electrodes may be configured to sense the ECAP signals. In some examples, the same electrodes may be configured to sense signals representative of transient movements of the patient. In other examples, other sensors, such as accelerometers, gyroscopes, or other movement sensors may be configured to sense movement of the patient that indicates the patient may have transitioned to a different posture state.

In some cases, stimulation delivery by cardiac device 152 obscures the evoked signal that IMD 110 senses. For example, the evoked signal (e.g., ECAP) is evoked in response to delivery of a stimulation signal. The evoked signal dissipates overtime, and may only be present in response to stimulation. That is, the evoked signal is not an intrinsic signal. Accordingly, sensing circuitry may be configured to sense the evoked signal within a time window that is relative to a time when stimulation is delivered that evokes the evoked signal that is sensed. The processing circuitry (e.g., of IMD 110) may receive information for a sensed evoked signal that is evoked due to stimulation delivery. For example, the processing circuitry (e.g., of IMD 110) may receive information for a sensed evoked signal that is sensed within a time window that is relative to a time when stimulation is delivered that evokes the evoked signal that is sensed. In accordance with one or more examples, the processing circuitry may be configured to determine whether noise generated from delivery of stimulation by a secondary medical device (e.g., cardiac device 152) is present in the sensed evoked signal.

Patient 105 is implanted with IMD 110 and cardiac device 152. However, it is possible the medical records are not properly updated to indicate that patient 105 is implanted with two different medical devices. Accordingly, as part of determining whether noise generated from delivery of stimulation by a secondary medical device (e.g., cardiac device 152) is present in the sensed evoked signal, the processing circuitry may detect the presence of an additional stimulation device, and provide an alert, if such information was not included in the medical record.

There may be various ways in which to determine whether noise generated from delivery of stimulation by a secondary medical device is present in the sensed evoked signal, described in more detail. The processing circuitry may be configured to perform various operations if determine that noise generated from delivery of stimulation by a secondary medical device is present in the sensed evoked signal. As one example, the processing circuitry may alter the stimulation and/or recording times (e.g., a time window when the evoked signal is sensed) so avoid recording concurrent electrical artifact generated from deliver of stimulation by the secondary medical device. For example, if cardiac device 152 stimulates every 61 seconds, and the sensing circuitry of IMD 110 is configured to sense the evoked signal every 20 ms at 50 Hz, after IMD 110 delivers of a stimulation signal, IMD 110 may delay or skip the stimulation signal so that the sensing of the evoked signal occurs at a time that is different than when cardiac device 152 delivers stimulation. In other words, the processing circuitry processing the sensed evoked signal based on a determination of whether the noise generated from delivery of stimulation by the secondary medical device is present in the sensed evoked signal includes changing a time when the evoked signal is to be sensed.

As another example, cardiac device 152 may be configured to alter a time when cardiac device 152 delivers stimulation. For example, assume IMD 110 is a pelvic health device (e.g., instead of an SCS device), and IMD 110 records signals (e.g., senses evoked signals) for 5 minutes a day. Cardiac device 152 may be configured to not delivery stimulation at times that overlap with when IMD 110 senses. Alternatively, if there are two closed-loop SCS devices implanted (one for upper limb pain and one for lower back pain), the processing circuitry of the SCS devices may coordinate timing of stimulation and sensing of evoked signals such that a secondary medical device is not delivering a pulse during the time that the primary medical device is sensing an evoked signal and vice versa, or the primary medical device is not sensing an evoked signal when the secondary medical device is delivering stimulation. This is another example way in which the processing circuitry may process the sensed evoked signal based on a determination of whether the noise generated from delivery of stimulation by the secondary medical device is present in the sensed evoked signal. Additional examples of processing the sensed evoked signals are described in more detail.

One example way in which the processing circuitry may determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal may be based on neural network techniques. For instance, in neural network techniques, such as ML/AI techniques, the processing circuitry may execute a trained model. Input to the trained model may be the sensed evoked signal. Output of the trained model may be information indicative of whether noise generated from delivery of stimulation by a secondary medical device is present in the sensed evoked signal.

In some examples, the trained model may be configured to process the sensed evoked signal based on a determination of whether the noise generated from delivery of stimulation by the secondary medical device is present in the sensed evoked signal. In some examples, the processing circuitry may be configured to process the sensed evoked signal based on a determination of whether the noise generated from delivery of stimulation by the secondary medical device is present in the sensed evoked signal, without or in combination with the trained model.

To generate the trained model (e.g., train the model to generate the trained model), model generation circuitry in one or more servers (not shown) may receive training data. To generate the training data, a medical professional may implant two medical devices in a subject (e.g., animal subject or volunteer patient). The medical professional may cause a first medical device of the two medical devices to evoke an evoked signal (e.g., output a stimulation that evokes the evoked signal). In one set of instances, the medical professional may ensure that the second medical device is not delivering stimulation. For these instances, the first device may sense the evoked signal, and store information of the sensed evoked signal, as stimulation-free sensed evoked signal.

In another set of instances, the medical professional may ensure that the second medical device is delivering stimulation during the time window in which the first medical device is sensing the evoked signal. For these instances, the first device may sense the evoked signal, and store information of the sensed evoked signal, as stimulation-present sensed evoked signal.

The model generation circuitry or the medical professional may compare the stimulation-present sensed evoked signal and the stimulation-free sensed evoked signal. The model generation circuitry or the medical professional may identify the noise in the stimulation-present sensed evoked signal based on the comparison. The model generation circuitry or the medical professional may tag the stimulation-present sensed evoked signal with information that identifies the noise.

The stimulation-present sensed evoked signal with the tags and the stimulation-free sensed evoked signal may be the training data and ground truths that are used to train the model. For instance, the model generation circuitry may train the model to identify the noise in the sensed evoked signal caused by the delivery of stimulation by another device, and confirm accuracy based on the tags.

In addition, the model generation circuitry may train the model to filter the noise from the stimulation-present sensed evoked signal to reconstruct the stimulation-free sensed evoked signal. The medical professional may confirm the accuracy of the filtering performed during the training phase by the model generation circuitry. Any inaccuracy may be fed back in as an error signal. The model generation circuitry may update parameters of the model based on the error signal. The medical professional may iterate over various training data using the above techniques, until the model generation circuitry generates a trained model that is configured to determine whether noise generated from delivery of stimulation by a secondary medical device is present in the sensed evoked signal. In this way, the processing circuitry may be configured to detect the sections of the sensed evoked signal that has concurrent artifact from electrical stimulation of a secondary medical device.

Using the above techniques, in some examples, but not necessarily all, the trained model may be configured to process the sensed evoked signal based on a determination of whether the noise generated from delivery of stimulation by the secondary medical device is present in the sensed evoked signal. For instance, as described the trained model may be configured to filter the noise generated from delivery of stimulation by a secondary medical device is present in the sensed evoked signal so that the resulting signal is the evoked signal without the noise. The processing circuitry may then use the resulting evoked signal without the noise for closed-loop stimulation. In this way, the processing circuitry may modify the sensed evoked signal or process the sensed evoked signal with the noise from the stimulation of the secondary medical device to separate out the actual evoked signal and the artifacts from the sensed evoked signal.

In some examples, if the noise is too large to be filtered, the processing circuitry may not perform filtering. In such examples, to process the sensed signal, the processing circuitry may be configured to avoid using the portion of the sensed evoked signal that has the noise, or may be configured to determine that this sensed evoked signal should not be used for controlling closed-loop stimulation.

Accordingly, to determine whether noise generated from delivery of stimulation by the secondary medical device (e.g., cardiac device 152) is present in the sensed evoked signal, the processing circuitry may be configured to execute a trained model that receives as input the information for the sensed evoked signal. As described, the trained model may be trained using training data that includes sensed signals where noise generate from another device is present in the sensed signals.

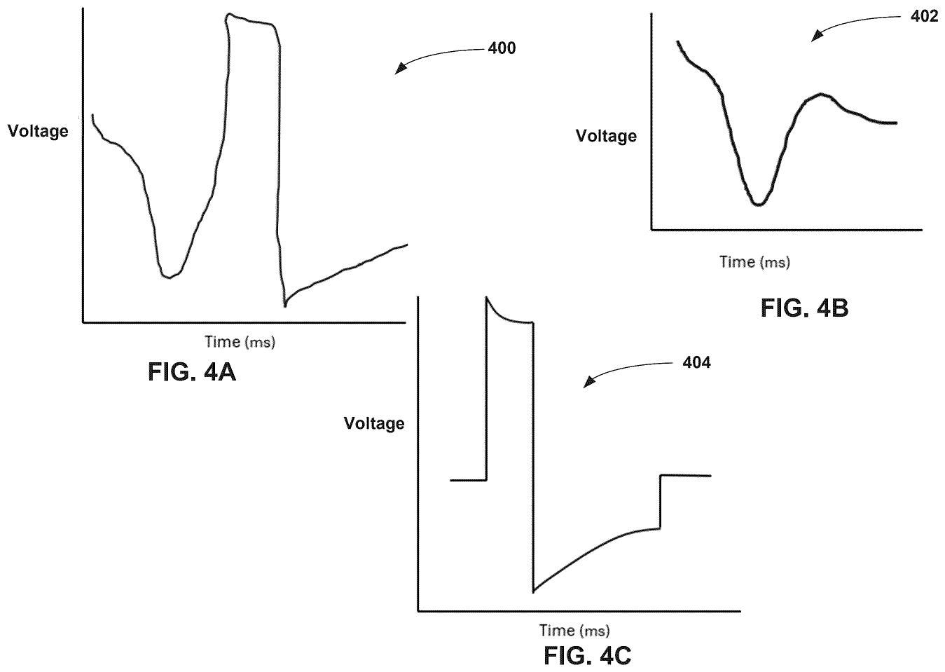

There may be various other techniques to determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal. Such example techniques are described in more detail below, such as with respect to FIGS. 4A, 4B, 5, 6A, and 6B, and elsewhere.

FIG. 2 is a block diagram illustrating an example configuration of components of an IMD 200, in accordance with one or more techniques of this disclosure. IMD 200 may be an example of IMD 110 of FIG. 1. In the example shown in FIG. 2, IMD 200 includes stimulation generation circuitry 202, switch circuitry 204, sensing circuitry 206, telemetry circuitry 208, processing circuitry 210, storage device 212, sensor(s) 222, and power source 224.

In the example shown in FIG. 2, storage device 212 stores patient data 240, stimulation parameter settings 242, and trained model 244 in separate memories within storage device 212 or separate areas within storage device 212. Patient data 240 may include parameter values, target characteristic values, or other information specific to the patient. In some examples, stimulation parameter settings 242 may include stimulation parameter values for respective different stimulation programs selectable by the clinician or patient for therapy. In this manner, each stored therapy stimulation program, or set of stimulation parameter values, of stimulation parameter settings 242 defines values for a set of electrical stimulation parameters (e.g., a stimulation parameter set), such as a stimulation electrode combination, electrode polarity, current or voltage amplitude, pulse width, pulse rate, and pulse shape, or duty cycle.

Storage device 212 may also store trained model 244, which processing circuitry 210 executes to determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal. Use of trained model 244 is not necessary in all examples, and is illustrated as one example way in which the techniques described in the disclosure may be performed.

In some examples, stimulation generation circuitry 202 generates electrical stimulation signals in accordance with the electrical stimulation parameters noted above. Other ranges of stimulation parameter values may also be useful and may depend on the target stimulation site within patient 105. While stimulation pulses are described, stimulation signals may be of any form, such as continuous-time signals (e.g., sine waves) or the like. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232, 234, or directed sensed signals from one or more of electrodes 232, 234 to sensing circuitry 206. In other examples, stimulation generation circuitry 202 and/or sensing circuitry 206 may include sensing circuitry to direct signals to and/or from one or more of electrodes 232, 234, which may or may not also include switch circuitry 204.

Sensing circuitry 206 is configured to monitor signals from any combination of electrodes 232, 234. In some examples, sensing circuitry 206 includes one or more amplifiers, filters, and analog-to-digital converters. Sensing circuitry 206 may be used to sense physiological signals, such as evoked signals (e.g., ECAP signals). In some examples, sensing circuitry 206 detects evoked signals from a particular combination of electrodes 232, 234. In some cases, the particular combination of electrodes for sensing evoked signals includes different electrodes than a set of electrodes 232, 234 used to deliver stimulation pulses. Alternatively, in other cases, the particular combination of electrodes used for sensing evoked signals includes at least one of the same electrodes as a set of electrodes used to deliver stimulation pulses to patient 105. Sensing circuitry 206 may provide signals to an analog-to-digital converter, for conversion into a digital signal for processing, analysis, storage, or output by processing circuitry 210.

Sensing circuitry 206 may be configured to sense the evoked signals within a particular time window. In general, a duration of the evoked signals may be limited before an amplitude of the evoked signals is not detectable. Accordingly, sensing circuitry 206 may sense the evoked signal within a time window that is relative to a time when stimulation is delivered that evokes the evoked signal that is sensed (e.g., sense for a 25 ms time window immediately after the delivery of the stimulation). Processing circuitry 210 may receive information for a sensed evoked signal that is evoked due to stimulation delivery. As one example, processing circuitry 210 may receive information for a sensed evoked signal that is sensed within a time window that is relative to a time when stimulation is delivered that evokes the evoked signal that is sensed.

Telemetry circuitry 208 supports wireless communication between IMD 200 and an external programmer (not shown in FIG. 2) or another computing device under the control of processing circuitry 210. Processing circuitry 210 of IMD 200 may receive, as updates to programs, values for various stimulation parameters such as amplitude and electrode combination, from the external programmer via telemetry circuitry 208. Processing circuitry 210 may store updates to the stimulation parameter settings 242 or any other data in storage device 212. Telemetry circuitry 208 in IMD 200, as well as telemetry circuits in other devices and systems described herein, such as the external programmer, may accomplish communication by radiofrequency (RF) communication techniques. In addition, telemetry circuitry 208 may communicate with an external medical device programmer (not shown in FIG. 2) via proximal inductive interaction of IMD 200 with the external programmer. The external programmer may be one example of external programmer 150 of FIG. 1. Accordingly, telemetry circuitry 208 may send information to the external programmer on a continuous basis, at periodic intervals, or upon request from IMD 110 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation signals according to stimulation parameter settings 242 and any other instructions stored in storage device 212 to apply stimulation parameter values specified by one or more of programs, such as amplitude, pulse width, pulse rate, and pulse shape of each of the stimulation signals.

In the example shown in FIG. 2, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 210 also controls stimulation generation circuitry 202 to generate and apply the stimulation signals to selected combinations of electrodes 232, 234. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord of the patient (not shown in FIG. 2) with selected electrodes 232, 234.

In other examples, however, stimulation generation circuitry 202 does not include a switch circuit and switch circuitry 204 does not interface between stimulation generation circuitry 202 and electrodes 232, 234. In these examples, stimulation generation circuitry 202 includes a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via switch circuitry 204 and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 206 is incorporated into a common housing with stimulation generation circuitry 202 and processing circuitry 210 in FIG. 2, in other examples, sensing circuitry 206 may be in a separate housing from IMD 200 and may communicate with processing circuitry 210 via wired or wireless communication techniques. In some examples, one or more of electrodes 232 and 234 are suitable for sensing the evoked signals (e.g., ECAPs). For instance, electrodes 232 and 234 may sense the voltage amplitude of a portion of the ECAP signals, where the sensed voltage amplitude, such as the voltage difference between features within the signal, is a characteristic the ECAP signal.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store patient data 240, stimulation parameter settings 242, and trained model 244.

In some examples, storage device 212 may store instructions on how processing circuitry 210 can adjust stimulation pulses in response to the determined characteristic values of the evoked signals. For example, processing circuitry 210 may monitor evoked signal characteristic values obtained from the evoked signals (or a signal derived from the evoked signals) to modulate stimulation parameter values (e.g., increase or decrease stimulation intensity to maintain a target therapeutic effect). In some examples, a target evoked signal characteristic value may vary for different situations for a patient, such as different posture states, times of day, activities, etc.

Sensor(s) 222 may include one or more sensing elements that sense values of a respective patient parameter, such as posture state. As described, electrodes 232 and 234 may be the electrodes that sense the characteristic value of the evoked signal. Sensor(s) 222 may include one or more accelerometers, optical sensors, chemical sensors, temperature sensors, pressure sensors, or any other types of sensors. Sensor(s) 222 may output patient parameter values that may be used as feedback to control delivery of therapy. For example, sensor(s) 222 may indicate patient activity, and processing circuitry 210 may increase the frequency of control pulses and evoked signal sensing in response to detecting increased patient activity. In one example, processing circuitry 210 may initiate control pulses and corresponding evoked signal sensing in response to a signal from sensor(s) 222 indicating that patient activity has exceeded an activity threshold. Conversely, processing circuitry 210 may decrease the frequency of control pulses and evoked signal sensing in response to detecting decreased patient activity. For example, in response to sensor(s) 222 no longer indicating that the sensed patient activity exceeds a threshold, processing circuitry 210 may suspend or stop delivery of control pulses and evoked signal sensing. In this manner, processing circuitry 210 may dynamically deliver control pulses and sense evoked signals based on patient activity to reduce power consumption of the system when the electrode-to-neuron distance is not likely to change and increase system response to evoked signal changes when electrode-to-neuron distance is likely to change. IMD 200 may include additional sensors within the housing of IMD 200 and/or coupled via one of leads 130 or other leads. In addition, IMD 200 may receive sensor signals wirelessly from remote sensors via telemetry circuitry 208, for example. In some examples, one or more of these remote sensors may be external to patient (e.g., carried on the external surface of the skin, attached to clothing, or otherwise positioned external to patient 105). In some examples, signals from sensor(s) 222 indicate a position or body state (e.g., sleeping, awake, sitting, standing, or the like), and processing circuitry 210 may select target evoked signal characteristic values according to the indicated position or body state.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries.

As described, processing circuitry 210 may be configured to perform the example techniques described in this disclosure. For instance, processing circuitry 210 may receive (e.g., from sensing circuitry 206) information for a sensed evoked signal that is sensed within a time window that is relative to a time when stimulation is delivered that evokes the evoked signal that is sensed. Processing circuitry 210 may determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal.

As one example, to determine whether noise generated from delivery of stimulation by the secondary medical device is present in the sensed evoked signal, processing circuitry 210 may be configured to execute trained model 244 that receives as input the information for the sensed evoked signal. As described, trained model 244 may be trained using training data that includes sensed signals where noise generate from another device is present in the sensed signals.

In some examples, to process the sensed evoked signal based on a determination that noise generated from delivery of stimulation by the secondary medical device is present in the sensed evoked signal, processing circuitry 210 may be configured to determine locations within the sensed evoked signal that corresponds to the noise generated from delivery of stimulation by the secondary medical device, and output the sensed evoked signal for closed-loop stimulation control except of samples of the sensed evoked signal that correspond to the determined locations. That is, in some examples, processing circuitry 210, using trained model 244, may be configured to determine portions of the sensed evoked signal that correspond to the noise generated by the delivery of the stimulation from the secondary medical device. Processing circuitry 210 may remove those portions, and rely on the remaining portions of the sensed evoked signal for closed-loop stimulation control. In this way, the portions of the sensed evoked signal that can lead to faulty changes in stimulation therapy may be removed.

In some examples, to process the sensed evoked signal based on a determination that noise generated from delivery of stimulation by the secondary medical device is present in the sensed evoked signal, processing circuitry 210, using trained model 244, may be configured to determine locations within the sensed evoked signal that corresponds to the noise generated from delivery of stimulation by the secondary medical device. Processing circuitry 210 may determine an estimate of the noise generated from delivery of stimulation by the secondary medical device, and subtract the estimate of the noise from samples of the sensed evoked signal that correspond to the determined locations. For example, trained model 244 may be trained to estimate of the noise generated from delivery of stimulation by the secondary medical device so that processing circuitry 210 may subtract the estimate of the noise. This way, the remaining signal is the evoked signal without the noise.

The above examples are described with respect to use of trained model 244. However, the example techniques are not so limited, and techniques in addition to or instead of neural network trained models may be used to determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal.

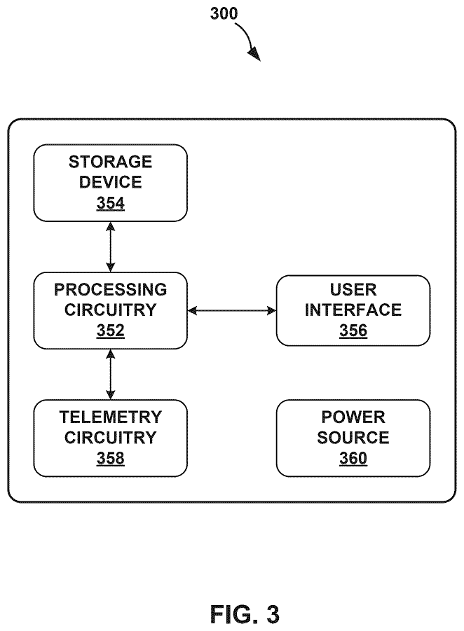

FIG. 3 is a block diagram illustrating an example configuration of components of an example external programmer 300. External programmer 300 may be an example of external programmer 150 of FIG. 1. Although external programmer 300 may generally be described as a hand-held device, external programmer 300 may be a larger portable device or a more stationary device. In addition, in other examples, external programmer 300 may be included as part of an external charging device or include the functionality of an external charging device. As illustrated in FIG. 3, external programmer 300 may include processing circuitry 352, storage device 354, user interface 356, telemetry circuitry 358, and power source 360. Storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include processing circuitry configured to perform the processes discussed with respect to processing circuitry 352.

In general, external programmer 300 includes any suitable arrangement of hardware, alone or in combination with software and/or firmware, to perform the techniques attributed to external programmer 300, and processing circuitry 352, user interface 356, and telemetry circuitry 358 of external programmer 300. In various examples, external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and telemetry circuitry 358 are described as separate modules, in some examples, processing circuitry 352 and telemetry circuitry 358 are functionally integrated. In some examples, processing circuitry 352 and telemetry circuitry 358 correspond to individual hardware units, such as ASICs, DSPs, FPGAs, or other hardware units.

Storage device 354 (e.g., a storage device) may store instructions that, when executed by processing circuitry 352, cause processing circuitry 352 and external programmer 300 to provide the functionality ascribed to external programmer 300 throughout this disclosure. For example, storage device 354 may include instructions that cause processing circuitry 352 to obtain a parameter set from memory, select a spatial electrode pattern, or receive a user input and send a corresponding command to IMD 200, or instructions for any other functionality. In addition, storage device 354 may include a plurality of programs, where each program includes a parameter set that defines therapy stimulation or control stimulation. Storage device 354 may also store data received from a medical device (e.g., IMD 110). For example, storage device 354 may store evoked signals related data recorded at a sensing circuitry 206, and storage device 354 may also store data from one or more sensors of the medical device.

User interface 356 may include a button or keypad, lights, a speaker for voice commands, a display, such as a liquid crystal (LCD), light-emitting diode (LED), or organic light-emitting diode (OLED). In some examples the display includes a touch screen. User interface 356 may be configured to display any information related to the delivery of electrical stimulation, identified posture states, sensed patient parameter values, or any other such information. User interface 356 may also receive user input (e.g., indication of when the patient perceives a stimulation pulse) via user interface 356. The input may be, for example, in the form of pressing a button on a keypad or selecting an icon from a touch screen. The input may request starting or stopping electrical stimulation, the input may request a new spatial electrode pattern or a change to an existing spatial electrode pattern, of the input may request some other change to the delivery of electrical stimulation.

Telemetry circuitry 358 may support wireless communication between the medical device and external programmer 300 under the control of processing circuitry 352. Telemetry circuitry 358 may also be configured to communicate with another computing device via wireless communication techniques, or direct communication through a wired connection. In some examples, telemetry circuitry 358 provides wireless communication via an RF or proximal inductive medium. In some examples, telemetry circuitry 358 includes an antenna, which may take on a variety of forms, such as an internal or external antenna.

Examples of local wireless communication techniques that may be employed to facilitate communication between external programmer 300 and IMD 110 include RF communication according to the 802.11 or Bluetooth® specification sets or other standard or proprietary telemetry protocols. In this manner, other external devices may be capable of communicating with external programmer 300 without needing to establish a secure wireless connection. As described herein, telemetry circuitry 358 may be configured to transmit a spatial electrode movement pattern or other stimulation parameter values to IMD 110 for delivery of electrical stimulation therapy. Although IMD 110 may determine whether noise generated from delivery of stimulation by a secondary medical device is present in the sensed evoked signal, and process the sensed evoked signal based on a determination of whether the noise generated from delivery of stimulation by the secondary medical device is present in the sensed evoked signal, in some examples, programmer 300 may perform these tasks alone or together with IMD 110 in a distributed function.

In some examples, selection of stimulation parameters or therapy stimulation programs are transmitted to the medical device for delivery to a patient (e.g., patient 105 of FIG. 1). In other examples, the therapy may include medication, activities, or other instructions that patient 105 must perform themselves or a caregiver perform for patient 105. In some examples, external programmer 300 provides visual, audible, and/or tactile notifications that indicate there are new instructions. External programmer 300 requires receiving user input acknowledging that the instructions have been completed in some examples.

User interface 356 of external programmer 300 may also be configured to receive an indication from a clinician instructing a processor of the medical device to update one or more therapy stimulation programs or to update the target characteristic values for the evoked signals. Updating therapy stimulation programs and target characteristic values may include changing one or more parameters of the stimulation pulses delivered by the medical device according to the programs, such as amplitude, pulse width, frequency, and pulse shape of the pulses and/or control pulses. User interface 356 may also receive instructions from the clinician commanding any electrical stimulation, including therapy stimulation and control stimulation to commence or to cease.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

FIG. 4A is a graph of example of a sensed evoked signal 400 that is sensed within a time window that is relative to a time when stimulation is delivered that evokes the evoked signal that is sensed. FIG. 4B is a graph of the actual evoked signal 402. FIG. 4C is a graph of an example template 404 used for determining whether noise generated from delivery of stimulation by a secondary medical device is present in the sensed evoked signal. As one example, template 404 may be similar to how a cardiac pacing pulse appears.

In one or more examples, a medical professional or some circuitry (e.g., template generation circuitry on a server) may access sensed evoked signals where stimulation from another device is delivered at a time that overlaps the time window during which the evoked signals are sensed. These sensed evoked signals may be from population data (e.g., from sensed evoked signals in patients with multiple implanted medical devices) or from test animal data.

Using the sensed evoked signals where stimulation from another device is delivered at a time that overlaps the time window during which the evoked signals are sensed, the template matching circuitry or the medical professional may generate one or more templates, like template 404. Each of the templates may be a trace of the sensed evoked signals, or a trace of an average of multiple sensed evoked signals. In general, the one or more templates may each represent how noise from a secondary medical device manifests in a sensed evoked signal.

In some examples, the templates may be based on the types of implanted medical devices. For example, one set of one or more templates may be for situations where the primary medical device is an SCS device, and the secondary medical device is a cardiac device. Another set of one or more templates may be for situations where the primary medical device is a pelvic therapy device, and the secondary medical device is an SCS device, and so forth.

Storage device 212 of IMD 200 may be configured to store the one or more templates, such as template 404. To determine whether noise generated from delivery of stimulation by the secondary medical device is present in the sensed evoked signal 400, processing circuitry 210 may be configured to access template 404, where template 404 represents a sensed signal where noise generated from delivery of stimulation is present in the sensed signal. Processing circuitry 201 may compare template 404 to the sensed evoked signal 400, and determine whether noise generated from delivery of stimulation by a secondary medical device (e.g., cardiac device 152) is present in the sensed evoked signal (e.g., sensed by IMD 110) based on the comparison. For example, sensed evoked signal 400 may be similar to template 404 with the actual evoked signal 402 added on.

For example, processing circuitry 210 may perform a sample-by-sample sum of absolute difference (SAD) calculation between sensed evoked signal 400 and template 404, and determine a SAD value. As another example, processing circuitry 210 may multiply sensed evoked signal 400 and template 404 together and then add the product. The result may be a single number (e.g., correlation value) that indicates how similar sensed evoked signal 400 and template 404 are.

Processing circuitry 210 may compare the SAD value or correlation value to a threshold value. If the SAD value is less than its threshold value or correlation value is greater than its threshold value, processing circuitry 210 may determine that noise generated from delivery of stimulation by the secondary medical device is present in the sensed evoked signal 400. If the SAD value is greater than its threshold value or the correlation value is less than its threshold value, processing circuitry 210 may determine that noise generated from delivery of stimulation by the secondary medical device is not present in the sensed evoked signal 400.

For instance, if the SAD value is relatively low or the correlation value is relatively high, that means that template 404 matches sensed evoked signal 400. If there is a match, processing circuitry 210 may determine that noise generated from delivery of stimulation by the secondary medical device is present in the sensed evoked signal 400. If there is no match, processing circuitry 210 may determine that noise generated from delivery of stimulation by the secondary medical device is not present in the sensed evoked signal 400.

In some examples, template 404 may be skewed relative to sensed evoked signal 400. That is, template 404 may not be aligned with sensed evoked signal 400. Accordingly, in some examples, processing circuitry 210 may determine multiple SAD values, where processing circuitry 210 determines a first SAD value based on a first alignment of template 404 and sensed evoked signal 400. Processing circuitry 210 may shift template 404, and recalculate the SAD value, and repeat such operations. Processing circuitry 210 may determine the minimum SAD value of the determined SAD values, and compare that minimum SAD value to the threshold value, as another way in which processing circuitry 210 may compare the SAD value to a threshold value. Also, if there are multiple templates, processing circuitry 210 may repeat such operations across multiple templates.

In one or more examples, during the template generation process, the medical professional or the template matching circuitry may tag portions in the templates that are the noise generated from the stimulation from another device. If processing circuitry 210 determines that noise generated from delivery of stimulation by the secondary medical device is present in the sensed evoked signal 400, processing circuitry 210 may determine which portions in sensed evoked signal 400 align with the tagged portions in the template 404. Processing circuitry 210 may determine that portions in sensed evoked signal 400 that aligned with the tagged portions in template 404 are portions of sensed evoked signal 400 where the noise generated from stimulation delivered by a secondary medical device is present.

Processing circuitry 210 may be configured to process the sensed evoked signal 400 based on a determination of whether the noise generated from delivery of stimulation by the secondary medical device is present in the sensed evoked signal. For example, processing circuitry 210 may determine locations (e.g., portions) within the sensed evoked signal 400 that corresponds to the noise generated from delivery of stimulation by the secondary medical device (e.g., such as using the tagged information of the templates). Processing circuitry 210 may output the sensed evoked signal 400 for closed-loop stimulation control except of samples of the sensed evoked signal that correspond to the determined locations.

As another example, to process the sensed evoked signal 400 based on a determination that noise generated from delivery of stimulation by the secondary medical device is present in the sensed evoked signal, the processing circuitry 210 may be configured to determine locations (e.g., portions) within the sensed evoked signal 400 that correspond to the noise generated from delivery of stimulation by the secondary medical device. Processing circuitry 210 may determine an estimate of the noise generated from delivery of stimulation by the secondary medical device. For example, the tagged information in template 404 may include information about how much noise was added to the sensed signal, which may be an estimate of the noise generated from delivery of stimulation by the secondary medical device. Processing circuitry 210 may subtract the estimate of the noise from samples of the sensed evoked signal that correspond to the determined locations.

In this way, processing circuitry 210 may detect the sections (e.g., locations or portions) of the sensed evoked signal 400 that has concurrent artifact (e.g., noise) from electrical stimulation of a secondary device. Processing circuitry 210 may determine whether sensed evoked signal 400 should be deleted or modified (e.g., whether portions of sensed evoked signal 400 should be removed or whether noise should be subtracted). That is, processing circuitry 210 may process the sensed evoked signal 400 with concurrent electrical artifact (e.g., noise) to separate out the sensed evoked signal from artifact. In some examples, processing circuitry 210 may delete sensed evoked signal 400, and not use any of it for closed-loop stimulation if the noise is too high. Processing circuitry 210 may keep a record of sensed evoked signals that were not used for closed-loop stimulation.

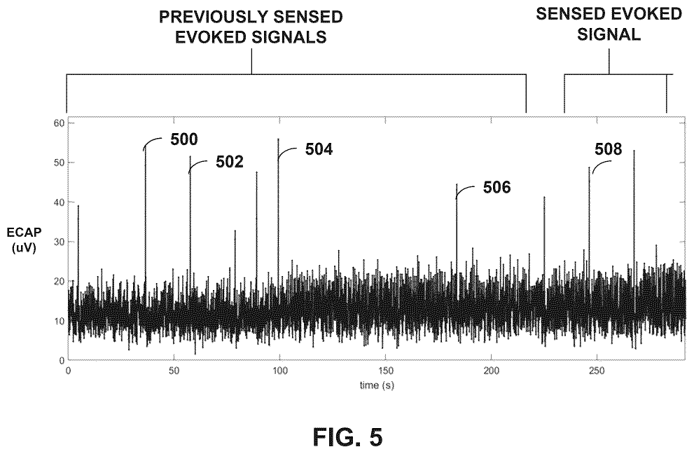

FIG. 5 is a graph of an example of a plurality of amplitude spikes in one or more instances of previously sensed evoked signals for determining whether the noise generated from delivery of stimulation by the secondary medical device is present in the sensed evoked signal. For certain types of medical devices, the stimulation delivered may be periodic (e.g., such as for pacemakers). In such examples, the noise generated from delivery of stimulation by a secondary medical device in the sensed evoked signal may be periodic, and manifest as an amplitude spike.

Stated another way, noise generated from delivery of stimulation by a secondary medical device may cause amplitude spikes in the sensed evoked signals. An amplitude spike may be considered as a relatively narrow portion of a sensed evoked signal where an amplitude is greater than a threshold. That is, the amplitude of the sensed evoked signal rises and falls quickly. In some cases, the stimulation from a secondary medical device may be periodic. Therefore, the noise generated from delivery of stimulation by the secondary medical device may be periodic.

For example, in FIG. 5, there are one or more previously sensed evoked signals, and within these one or more previously evoked signals there are amplitude spikes 500, 502, 504, and 506. Each of amplitude spikes 500, 502, 504, and 506 occur at respective time stamps (e.g., the times at which spikes 500, 502, 504, and 506 occurred). Processing circuitry 210 may utilize the time stamps to determine whether spikes 500, 502, 504, and 506 are occurring on a periodic basis. For instance, processing circuitry 210 may determine whether a constant time passes between spikes 500, 502, 504, and 506. Spikes 500, 502, 504, and 506 are called out for ease, and there may be more or fewer spikes.

If spikes 500, 502, 504, and 506 are occurring on a periodic basis, it is likely that such noise (e.g., artifacts) are due to delivery of stimulation by a secondary medical device because such noise is deterministic and not random. Based on the period at which spikes 500, 502, 504, and 506 occur, processing circuitry 210 may estimate a time stamp when the next spike is to occur.

Processing circuitry 210 may determine whether there is an amplitude spike, like amplitude spike 508, at the estimated time stamp. If there is an amplitude spike at the estimated time stamp, processing circuitry 210 may determine that such noise is from a secondary medical device.

Processing circuitry 210 may then process the sensed evoked signal. For instance, processing circuitry 210 may remove the portions of the sensed evoked signal that corresponds to where the amplitude spike is present in the sensed evoked signal. As another example, processing circuitry 210 may estimate how much noise the amplitude spike is adding, and subtract that noise from the sensed evoked signal.

Accordingly, processing circuitry 210 may determine a plurality of amplitude spikes 500, 502, 504, and 506 in one or more instances of previously sensed evoked signals, as illustrated in FIG. 5. An amplitude spike may be a relatively narrow portion of the one or more instances of the previously evoked signals where an amplitude of the one or more previously evoked signals is greater than a threshold. For example, amplitude spikes 500, 502, 504, and 506 occur over a narrow portion where the amplitude rises and fall relatively quickly. Processing circuitry 210 may determine whether the plurality of amplitude spikes 500, 502, 504, and 506 are periodic, and determine whether the noise generated from delivery of stimulation by the secondary medical device is present in the sensed evoked signal based on whether the plurality of amplitude spikes are periodic. For instance, processing circuitry 210 may estimate where a next amplitude spike will be, and if there is an amplitude spike in the sensed evoked signal, processing circuitry 210 may determine that such an amplitude spike is caused by noise. Also, in some examples, processing circuitry 210 may automatically determine that the portion where the next spike is estimated to be in the sensed evoked signal is noise without necessarily confirming that an amplitude spike is present in the portion.

In one or more examples, there may be benefits in utilizing previously sensed evoked signals to determine whether there is noise the sensed evoked signal. The time window within which the sensed evoked signal is sensed may be relatively short. Therefore, there may not be multiple amplitude spikes or an insufficient number of amplitude spikes to determine periodicity.

Accordingly, FIG. 5 also illustrates example techniques in which processing circuitry 210 may detect the sections (e.g., locations or portions) of the sensed evoked signal that has concurrent artifact from electrical stimulation of a secondary device (e.g., based on an estimation of where a next amplitude spike will be if the amplitude spikes in the previously sensed evoked signals are periodic). Processing circuitry 210 may determine whether the sensed evoked signal with electrical stimulation should be deleted/modified, as part of processing the sensed evoked signal (e.g., remove the portions of the sensed evoked signal located where the noise is present or subtract the noise). That is, processing circuitry 210 may process the sensed evoked signal with concurrent electrical artifact (e.g., noise) to separate out the evoked signal from the artifact.

In the example of FIG. 5, processing circuitry 210 relied on previously sensed evoked signals. However, the techniques are not so limited. As described, evoked signals are sensed within a time window that is relative to a time when stimulation is delivered that evokes the evoked signal that is sensed (e.g., the time window is immediately after the stimulation that evokes the evoked signal). However, in some examples, in addition to the evoked signal, sensing circuitry 206 may be configured to sense signals in a continuous mode.

As one example, using the same electrodes used for sensing the evoked signal or different electrodes, sensing circuitry 206 may be configured to sense local field potentials (LFPs). Sensing circuitry 206 may sense LFPs or other intrinsic signals in continuous mode (e.g., continuously sensed LFPs or other intrinsic signals) because LPFs and other intrinsic signals are generated by patient 105, and are not generated because of delivery of stimulation. If there is noise generated by delivery of stimulation from a secondary medical device, such noise would also be present on the sensed signal that is sensed in continuous mode.

In one or more examples, processing circuitry 210 may receive information of a sensed signal that is sensed in a continuous mode (e.g., receive information indicative of an LFP from sensing circuitry 206). Processing circuitry 210 may determine whether noise generated from delivery of the stimulation by the secondary medical device is present in the sensed signal that is sensed in the continuous mode. For example, processing circuitry 210 may determine whether there are periodic amplitude spikes in the sensed signal that is sensed in the continuous mode, similar to the description above for previously sensed evoked signals, to determine whether noise generated from delivery of the stimulation by the secondary medical device is present in the sensed signal that is sensed in the continuous mode.

Processing circuitry 210 may determine whether the noise generated from delivery of stimulation by the secondary medical device is present in the sensed evoked signal based on the determination of whether noise generated from delivery of the stimulation by the secondary medical device is present in the sensed signal that is sensed in the continuous mode. Processing circuitry 210 may then process the sensed evoked signal. For instance, processing circuitry 210 may determine locations of the noise in the sensed evoked signal. As an example, processing circuitry 210 may determine locations of the noise in the sensed signal that is sensed in continuous mode, and determine corresponding locations in the sensed evoked signal. Processing circuitry 210 may remove the determined location in the sensed evoked signal for use in closed-loop stimulation.

In some examples, processing circuitry 210 may estimate the noise in the sensed evoked signal based on the noise in the sensed signal sensed in continuous mode. For instance, the amplitude of the spikes in the sensed signal sensed in continuous mode may be an estimate (e.g., after scaling) of the noise in the sensed evoked signal. Processing circuitry 210 may subtract of the estimate of the noise from the locations in the sensed evoked signal where the noise is present.

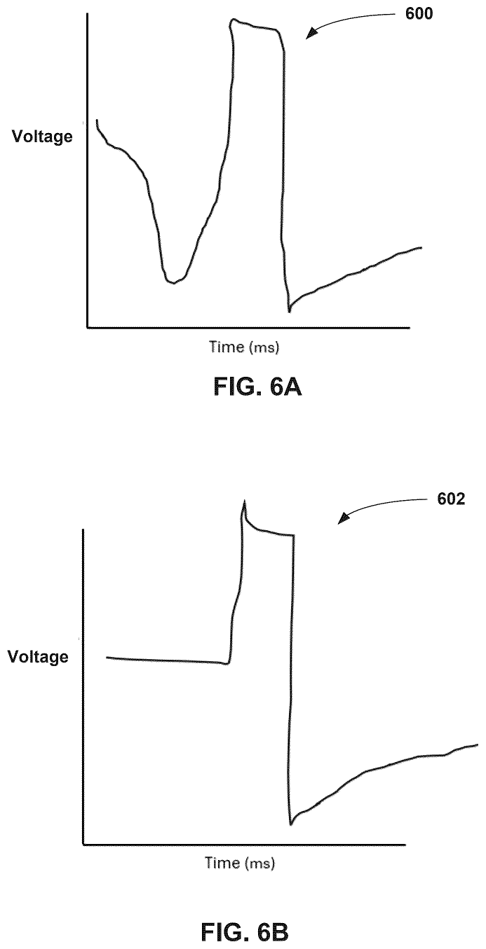

FIG. 6A is a graph of example of a first sensed evoked signal 600 that is sensed with a first sensing channel. FIG. 6B is a graph of example of a second sensed evoked signal 602 that is sensed with a second sensing channel. A sensing channel refers to the electrodes used to sense the evoked signal. The first and second sensing channels may be different (e.g., different electrodes are used for sensing) but both are sensing channels for IMD 200. Processing circuitry 210 may be configured to determine whether noise generated from delivery of stimulation by a secondary medical device is present in the sensed evoked signal based on whether the first sensed evoked signal 600 and the second evoked signal 602 are similar.

In one or more examples, first sensed evoked signal 600 and second sensed evoked signal 602 may be sensed during the same time window relative to when stimulation is delivered. Assume that first sensed evoked signal 600 is the sensed evoked signal that is used for closed-loop stimulation. If there is no noise due to the stimulation from another device, first sensed evoked signal 600 and second sensed evoked signal 602 should be different. This is because first sensed evoked signal 600 should be measuring the actual neuron propagation since the electrodes of the first sensing channel may be closer to the nerves used for closed-loop stimulation compared to the electrodes of the second sensing channel.

However, if first sensed evoked signal 600 and second sensed evoked signal 602 are similar, at least in certain locations (e.g., portions), then both the first sensing channel and the second sensing channel are sensing the same noise, and the source of that noise is relatively distant from IMD 200, and therefore likely or possibly from stimulation delivered by a secondary medical device. Accordingly, processing circuitry 210 may receive information for a first sensed evoked signal 600 that is sensed using a first sensing channel within a time window that is relative to a time when stimulation is delivered that evokes the first evoked signal 600 that is sensed by the first sensing channel, and receive information for a second sensed evoked signal 602 that is sensed using a second sensing channel within the time window that is relative to the time when stimulation is delivered that evokes the second evoked signal 602 that is sensed by the second sensing channel.

Processing circuitry 210 may determine a similarity value indicative of a similarity between the first sensed evoked signal 600 and the second sensed evoked signal 602, and determine whether the noise generated from delivery of stimulation by the secondary medical device is present in the sensed evoked signal based on the similarity value. For example, if first sensed evoked signal 600 and second evoked signal 602 are similar, there is a possibility that noise generated from delivery of stimulation by the secondary medical device is present in the sensed evoked signal.

There may be various ways to determine similarity value. One example is a SAD value between first sensed evoked signal 600 and second sensed evoked signal 602, as described above. Another example may be a correlation value, as described above, such as processing circuitry 210 multiplying first sensed evoked signal 600 and second sensed evoked signal 602 and then adding the product to result in a number that indicates how similar first sensed evoked signal 600 and second sensed evoked signal 602 are.

In the above example, processing circuitry 210 may determine that noise generated from delivery of stimulation by a secondary medical device is present if first sensed evoked signal 600 and second sensed evoked signal 602 are similar. However, in some examples, it is possible that that noise generated from delivery of stimulation by a secondary medical device is present even if first sensed evoked signal 600 and second sensed evoked signal 602 are not similar. For instance, if the secondary medical device is closer to the first sensing channel than the second sensing channel, there is a possibility that there will be much more noise on first evoked sensed signal 600 than second evoked sensed signal 602 making the first sensed evoked signal 600 and second sensed evoked signal 602 dissimilar even though there is noise present on first sensed evoked signal 600.

Accordingly, in some examples, determining similarity between evoked sensed signals on different sensing channels may be used in combination with other techniques to determine whether noise generated from delivery of stimulation by a secondary medical device is present in the sensed evoked signal. However, it may be possible to determine similarity between evoked sensed signals on different sensing channels as the sole or primary indicator of whether noise generated from delivery of stimulation by a secondary medical device is present in the sensed evoked signal.

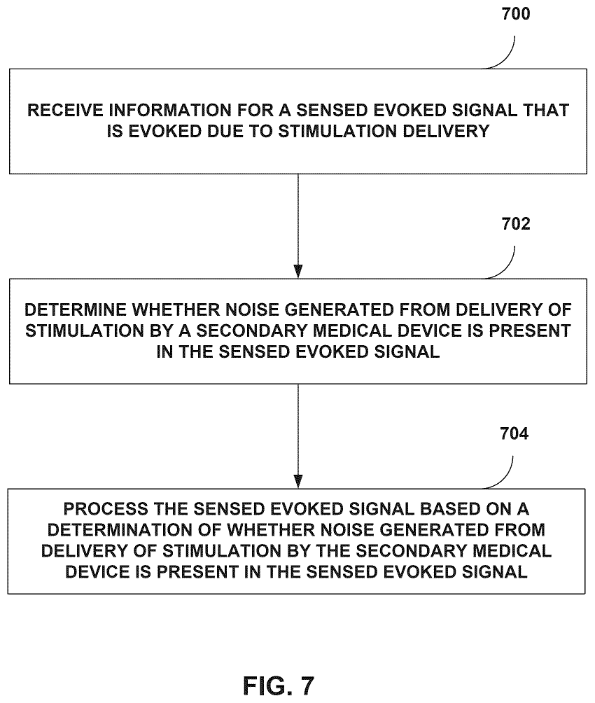

FIG. 7 is a flow chart illustrating an example method of operation, in accordance with one or more techniques of this disclosure. For ease, the example techniques are described with respect to processing circuitry 210, but the techniques may be performed by other processing circuitry or processing circuitry in combination with processing circuitry 210.

Processing circuitry 210 may receive information for a sensed evoked signal that is evoked due to stimulation delivery (700). As one example, processing circuitry 210 may receive information for a sensed evoked signal that is sensed within a time window that is relative to a time when stimulation is delivered that evokes the evoked signal that is sensed. For example, stimulation generation circuitry 202 may be configured to deliver stimulation that evokes an evoked signal. Sensing circuitry 206 may be configured to sense the evoked signal in a time window that immediately follows the delivery of the stimulation using one or more electrodes 232, 234.

In some examples, the sensed evoked signal is a sensed ECAP. In some examples, the sensed evoked signal is sensed along a spinal cord 120 of a patient 105. However, the techniques are applicable generally to evoked signals that are sensed in a time window, and should not be considered limited to ECAPs or evoked signals sensed along spinal cord 120.

Processing circuitry 210 may determine whether noise generated from delivery of stimulation by a secondary medical device is present in the sensed evoked signal (702). There may be various ways in which processing circuitry 210 may determine whether noise generated from delivery of stimulation by a secondary medical device is present in the sensed evoked signal. The following are a few examples that may be used separately or in any combination, and are described with respect to FIGS. 4A, 4B, 5, 6A, and 6B, and additional description.

As one example, to determine whether noise generated from delivery of stimulation by the secondary medical device is present in the sensed evoked signal, processing circuitry 210 may be configured to execute a trained model 244 that receives as input the information for the sensed evoked signal. Trained model 244 may be trained using training data that includes sensed signals where noise generate from another device is present in the sensed signals.

As another example, to determine whether noise generated from delivery of stimulation by the secondary medical device is present in the sensed evoked signal, processing circuitry 210 may be configured to access a template 404 (e.g., from storage device 212), the template 404 representing a sensed signal where noise generated from delivery of stimulation is present in the sensed signal. Processing circuitry 210 may compare the template 404 to the sensed evoked signal 400 (e.g., determine SAD value or correlation value). Processing circuitry 210 may determine whether noise generated from delivery of stimulation by a secondary medical device is present in the sensed evoked signal based on the comparison.

As another example, to determine whether the noise generated from delivery of stimulation by the secondary medical device is present in the sensed evoked signal, processing circuitry 210 may be configured to determine a plurality of amplitude spikes 500, 502, 504, and 506 in one or more instances of previously sensed evoked signals. An amplitude spike may be a relatively narrow portion of the one or more instances of the previously evoked signals where an amplitude of the one or more previously evoked signals is greater than a threshold. Processing circuitry 210 may determine whether the plurality of amplitude spikes 500, 502, 504, and 506 are periodic, and determine whether the noise generated from delivery of stimulation by the secondary medical device is present in the sensed evoked signal based on whether the plurality of amplitude spikes 500, 502, 504, and 506 are periodic. For instance, processing circuitry 210 may determine a location of amplitude spike 508 in the sensed evoked signal. If there actually is an amplitude spike 508 in the sensed evoked signal, then there is noise from the delivery of stimulation by the secondary medical device.

As another example, to determine whether the noise generated from delivery of stimulation by the secondary medical device is present in the sensed evoked signal, the processing circuitry 210 may be configured to receive information of a sensed signal that is sensed in a continuous mode (e.g., an LFP). Processing circuitry 210 may determine whether noise generated from delivery of the stimulation by the secondary medical device is present in the sensed signal that is sensed in the continuous mode (e.g., such as determining amplitude spikes in the LFP and whether the amplitude spikes are periodic), and determine whether the noise generated from delivery of stimulation by the secondary medical device is present in the sensed evoked signal based on the determination of whether noise generated from delivery of the stimulation by the secondary medical device is present in the sensed signal that is sensed in the continuous mode.

As another example, the sensed evoked signal may be a first sensed evoked signal 600 sensed using a first sensing channel. In such examples, to determine whether the noise generated from delivery of stimulation by the secondary medical device is present in the sensed evoked signal, processing circuitry 210 may be configured to receive information for a second sensed evoked signal 602 that is sensed using a second sensing channel. Processing circuitry 210 may determine a similarity value (e.g., SAD value or correlation value) indicative of a similarity between the first sensed evoked signal 600 and the second sensed evoked signal 602. Processing circuitry 210 may determine whether the noise generated from delivery of stimulation by the secondary medical device is present in the sensed evoked signal based on the similarity value. For example, if first sensed evoked signal 600 and the second sensed evoked signal 602 are similar, there may be noise from a secondary medical device.

Processing circuitry 210 process the sensed evoked signal based on a determination of whether the noise generated from delivery of stimulation by the secondary medical device is present in the sensed evoked signal (704). As one example, to process the sensed evoked signal based on a determination that noise generated from delivery of stimulation by the secondary medical device is present in the sensed evoked signal, the processing circuitry 210 may be configured to determine locations within the sensed evoked signal that corresponds to the noise generated from delivery of stimulation by the secondary medical device, and output the sensed evoked signal for closed-loop stimulation control except of samples of the sensed evoked signal that correspond to the determined locations.

As another example, to process the sensed evoked signal based on a determination that noise generated from delivery of stimulation by the secondary medical device is present in the sensed evoked signal, the processing circuitry 210 may be configured to determine locations within the sensed evoked signal that correspond to the noise generated from delivery of stimulation by the secondary medical device. Processing circuitry 210 may determine an estimate of the noise generated from delivery of stimulation by the secondary medical device, and subtract the estimate of the noise from samples of the sensed evoked signal that correspond to the determined locations.

As additional examples, processing circuitry 210 may alter the stimulation and/or recording times (e.g., a time window when the evoked signal is sensed) so avoid recording concurrent electrical artifact generated from deliver of stimulation by the secondary medical device. As another example, the secondary medical device may be configured to alter a time when the secondary medical device delivers stimulation.

There may be other actions that processing circuitry 210 undertakes based on a determination that noise generated from delivery of stimulation by a secondary medical device is present in the sensed evoked signal. For example, based on the sensed evoked signal, processing circuitry 210 may adjust the stimulation parameter settings 242 of the stimulation that stimulation generation circuitry 202 delivers. Processing circuitry 210 may compare the sensed evoked signal to one or more thresholds. One threshold may be a reaction threshold (e.g., upper threshold), and another threshold may be a recovery threshold (e.g., lower threshold). If the sensed evoked signal is greater than the reaction threshold, then processing circuitry 210 may lower amplitude, as one example. If the sensed evoked signal is less than the reaction threshold, then processing circuitry 210 may increase amplitude, as one example.

The rate of this change in stimulation parameter, amplitude being one example, is referred to as the recovery speed. The rate of this increase or decrease (e.g., recovery speed) has a default rate, but may be changed. The recovery speed may be set in a way that is appropriate for the condition. For example, if someone does a back arch/cough/laugh, that the increase in stimulation is a smooth experience, meaning that the recovery speed is relatively low. However, this means if processing circuitry 210 changes amplitude based on the sensed evoked signal that includes noise (e.g., from a pacemaker pulse), it may take a second, as an example, to recover back to the target amplitude. If the pacemaker pulse is every second, then it may take too long or may processing circuitry 210 may never be able to adjust the amplitude back to the target amplitude.

In one or more examples, processing circuitry may automatically adjust parts of the algorithm to recover faster to the nominal stimulation. If the amplitude spike is deemed physiological (e.g., not due to stimulation from secondary medical device), then processing circuitry 210 may adjust the stimulation parameter with the normal recovery speed. If the amplitude spike is deemed noise from a secondary device, then processing circuitry 210 may adjust either at a faster rate and/or returns to the amplitude delivered immediately before the spike.

Accordingly, in one or more examples, to process the sensed evoked signal based on a determination of whether the noise generated from delivery of stimulation by the secondary medical device is present in the sensed evoked signal, processing circuitry 210 may be configured to determine a rate at which to adjust stimulation parameters. For instance, if the noise generated from delivery of stimulation by the secondary medical device is present in the sensed evoked signal, processing circuitry 210 may use the sensed evoked signal to determine to increase the recovery rate (e.g., increase how quickly processing circuitry 210 changes the stimulation parameters). If the noise generated from delivery of stimulation by the secondary medical device is not present in the sensed evoked signal, processing circuitry 210 may use the sensed evoked signal to determine to not change the recovery rate.

For example, there may be set step sizes that processing circuitry 210 may use to increase or decrease the stimulation parameters. For fast recovery speed, processing circuitry 210 may increase or decrease the stimulation parameters at a first step size. For normal recovery speed, processing circuitry 210 may increase or decrease the stimulation parameters at a second step size. The first step size may be greater than the second step size.

The following examples are described herein, and may be performed separately or in any combination.

Example 1: A medical device comprising: processing circuitry configured to: receive information for a sensed evoked signal that is evoked due to stimulation delivery; determine whether noise generated from delivery of stimulation by a secondary medical device is present in the sensed evoked signal; and process the sensed evoked signal based on the determination of whether the noise generated from delivery of stimulation by the secondary medical device is present in the sensed evoked signal.

Example 2: The medical device of example 1, wherein to determine whether the noise generated from delivery of stimulation by the secondary medical device is present in the sensed evoked signal, the processing circuitry is configured to execute a trained model that receives as input the information for the sensed evoked signal, wherein the trained model is trained using training data that includes sensed signals where noise generate from another device is present in the sensed signals.

Example 3. The medical device of any of examples 1 and 2, wherein to determine whether the noise generated from delivery of stimulation by the secondary medical device is present in the sensed evoked signal, the processing circuitry is configured to: access a template, the template representing a sensed signal where noise generated from delivery of stimulation is present in the sensed signal; compare the template to the sensed evoked signal; and determine whether noise generated from delivery of stimulation by a secondary medical device is present in the sensed evoked signal based on the comparison.

Example 4. The medical device of any of examples 1-3, wherein to determine whether the noise generated from delivery of stimulation by the secondary medical device is present in the sensed evoked signal, the processing circuitry is configured to: determine a plurality of amplitude spikes in one or more instances of previously sensed evoked signals, an amplitude spike comprising a relatively narrow portion of the one or more instances of the previously evoked signals where an amplitude of the one or more previously evoked signals is greater than a threshold; determine whether the plurality of amplitude spikes are periodic; and determine whether the noise generated from delivery of stimulation by the secondary medical device is present in the sensed evoked signal based on whether the plurality of amplitude spikes are periodic.

Example 5. The medical device of any of examples 1-4, wherein the sensed evoked signal comprises a first sensed evoked signal sensed using a first sensing channel, wherein to determine whether the noise generated from delivery of stimulation by the secondary medical device is present in the sensed evoked signal, the processing circuitry is configured to: receive information for a second sensed evoked signal that is sensed using a second sensing channel; determine a similarity value indicative of a similarity between the first sensed evoked signal and the second sensed evoked signal; and determine whether the noise generated from delivery of stimulation by the secondary medical device is present in the sensed evoked signal based on the similarity value.

Example 6. The medical device of any of examples 1-5, wherein to determine whether the noise generated from delivery of stimulation by the secondary medical device is present in the sensed evoked signal, the processing circuitry is configured to: receive information of a sensed signal that is sensed in a continuous mode; determine whether noise generated from delivery of the stimulation by the secondary medical device is present in the sensed signal that is sensed in the continuous mode; and determine whether the noise generated from delivery of stimulation by the secondary medical device is present in the sensed evoked signal based on the determination of whether noise generated from delivery of the stimulation by the secondary medical device is present in the sensed signal that is sensed in the continuous mode.

Example 7. The medical device of any of examples 1-6, wherein to process the sensed evoked signal based on a determination that noise generated from delivery of stimulation by the secondary medical device is present in the sensed evoked signal, the processing circuitry is configured to: determine locations within the sensed evoked signal that corresponds to the noise generated from delivery of stimulation by the secondary medical device; and output the sensed evoked signal for closed-loop stimulation control except of samples of the sensed evoked signal that correspond to the determined locations.

Example 8. The medical device of any of examples 1-6, wherein to process the sensed evoked signal based on a determination that noise generated from delivery of stimulation by the secondary medical device is present in the sensed evoked signal, the processing circuitry is configured to: determine locations within the sensed evoked signal that correspond to the noise generated from delivery of stimulation by the secondary medical device; determine an estimate of the noise generated from delivery of stimulation by the secondary medical device; and subtract the estimate of the noise from samples of the sensed evoked signal that correspond to the determined locations.

Example 9. The medical device of any of examples 1-8, wherein to process the sensed evoked signal based on the determination of whether the noise generated from delivery of stimulation by the secondary medical device is present in the sensed evoked signal, the processing circuitry is configured to determine a rate at which to adjust stimulation parameters.

Example 10. The medical device of any of examples 1-9, wherein the sensed evoked signal comprises a sensed evoked compound action potential (ECAP) that is sensed along a spinal cord of a patient.

Example 11. A method for noise detection, the method comprising: receive information for a sensed evoked signal that is evoked due to stimulation delivery; determining whether noise generated from delivery of stimulation by a secondary medical device is present in the sensed evoked signal; and processing the sensed evoked signal based on the determination of whether the noise generated from delivery of stimulation by the secondary medical device is present in the sensed evoked signal.

Example 12. The method of example 11, wherein determining whether the noise generated from delivery of stimulation by the secondary medical device is present in the sensed evoked signal comprises executing a trained model that receives as input the information for the sensed evoked signal, wherein the trained model is trained using training data that includes sensed signals where noise generate from another device is present in the sensed signals.

Example 13. The method of any of examples 11 and 12, wherein determining whether the noise generated from delivery of stimulation by the secondary medical device is present in the sensed evoked signal comprises: accessing a template, the template representing a sensed signal where noise generated from delivery of stimulation is present in the sensed signal; comparing the template to the sensed evoked signal; and determining whether noise generated from delivery of stimulation by a secondary medical device is present in the sensed evoked signal based on the comparison.

Example 14. The method of any of examples 11-13, wherein determining whether the noise generated from delivery of stimulation by the secondary medical device is present in the sensed evoked signal comprises: determining a plurality of amplitude spikes in one or more instances of previously sensed evoked signals, an amplitude spike comprising a relatively narrow portion of the one or more instances of the previously evoked signals where an amplitude of the one or more previously evoked signals is greater than a threshold; determining whether the plurality of amplitude spikes are periodic; and determining whether the noise generated from delivery of stimulation by the secondary medical device is present in the sensed evoked signal based on whether the plurality of amplitude spikes are periodic.

Example 15. The method of any of examples 11-14, wherein the sensed evoked signal comprises a first sensed evoked signal sensed using a first sensing channel, wherein determining whether the noise generated from delivery of stimulation by the secondary medical device is present in the sensed evoked signal comprises: receiving information for a second sensed evoked signal that is sensed using a second sensing channel; determining a similarity value indicative of a similarity between the first sensed evoked signal and the second sensed evoked signal; and determining whether the noise generated from delivery of stimulation by the secondary medical device is present in the sensed evoked signal based on the similarity value.

Example 16. The method of any of examples 11-15, wherein determining whether the noise generated from delivery of stimulation by the secondary medical device is present in the sensed evoked signal comprises: receiving information of a sensed signal that is sensed in a continuous mode; determining whether noise generated from delivery of the stimulation by the secondary medical device is present in the sensed signal that is sensed in the continuous mode; and determining whether the noise generated from delivery of stimulation by the secondary medical device is present in the sensed evoked signal based on the determination of whether noise generated from delivery of the stimulation by the secondary medical device is present in the sensed signal that is sensed in the continuous mode.

Example 17. The method of any of examples 11-16, wherein processing the sensed evoked signal based on a determination that noise generated from delivery of stimulation by the secondary medical device is present in the sensed evoked signal comprises: determining locations within the sensed evoked signal that corresponds to the noise generated from delivery of stimulation by the secondary medical device; and outputting the sensed evoked signal for closed-loop stimulation control except of samples of the sensed evoked signal that correspond to the determined locations.

Example 18. The method of any of examples 11-16, wherein processing the sensed evoked signal based on a determination that noise generated from delivery of stimulation by the secondary medical device is present in the sensed evoked signal comprises: determining locations within the sensed evoked signal that correspond to the noise generated from delivery of stimulation by the secondary medical device; determining an estimate of the noise generated from delivery of stimulation by the secondary medical device; and subtracting the estimate of the noise from samples of the sensed evoked signal that correspond to the determined locations.

Example 19. The method of any of examples 11-18, wherein processing the sensed evoked signal based on a determination of whether the noise generated from delivery of stimulation by the secondary medical device is present in the sensed evoked signal comprises determining a rate at which to adjust stimulation parameters.

Example 20. The method of any of examples 11-19, wherein the sensed evoked signal comprises a sensed evoked compound action potential (ECAP) that is sensed along a spinal cord of a patient.

Example 21. A computer-readable storage medium storing instructions thereon that when executed cause one or more processors to perform the method of any one or combination of examples 11-20.

Example 22. A system comprising means for performing the method of any one or combination of examples 11-20.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. For example, processing circuitry may conduct processing off-line and conduct automatic checks of patient ECAP signals and update programming from a remote location. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A medical device comprising:
processing circuitry configured to:
receive information for a sensed evoked signal that is evoked due to stimulation delivery;
determine whether noise generated from delivery of stimulation by a secondary medical device is present in the sensed evoked signal; and
process the sensed evoked signal based on the determination of whether the noise generated from delivery of stimulation by the secondary medical device is present in the sensed evoked signal.

2. The medical device of claim 1, wherein to determine whether the noise generated from delivery of stimulation by the secondary medical device is present in the sensed evoked signal, the processing circuitry is configured to execute a trained model that receives as input the information for the sensed evoked signal, wherein the trained model is trained using training data that includes sensed signals where noise generate from another device is present in the sensed signals.

3. The medical device of claim 1, wherein to determine whether the noise generated from delivery of stimulation by the secondary medical device is present in the sensed evoked signal, the processing circuitry is configured to:
access a template, the template representing a sensed signal where noise generated from delivery of stimulation is present in the sensed signal;
compare the template to the sensed evoked signal; and
determine whether noise generated from delivery of stimulation by a secondary medical device is present in the sensed evoked signal based on the comparison.

4. The medical device of claim 1, wherein to determine whether the noise generated from delivery of stimulation by the secondary medical device is present in the sensed evoked signal, the processing circuitry is configured to:
determine a plurality of amplitude spikes in one or more instances of previously sensed evoked signals, an amplitude spike comprising a relatively narrow portion of the one or more instances of the previously evoked signals where an amplitude of the one or more previously evoked signals is greater than a threshold;
determine whether the plurality of amplitude spikes are periodic; and
determine whether the noise generated from delivery of stimulation by the secondary medical device is present in the sensed evoked signal based on whether the plurality of amplitude spikes are periodic.

5. The medical device of claim 1, wherein the sensed evoked signal comprises a first sensed evoked signal sensed using a first sensing channel, wherein to determine whether the noise generated from delivery of stimulation by the secondary medical device is present in the sensed evoked signal, the processing circuitry is configured to:
receive information for a second sensed evoked signal that is sensed using a second sensing channel;
determine a similarity value indicative of a similarity between the first sensed evoked signal and the second sensed evoked signal; and
determine whether the noise generated from delivery of stimulation by the secondary medical device is present in the sensed evoked signal based on the similarity value.

6. The medical device of claim 1, wherein to determine whether the noise generated from delivery of stimulation by the secondary medical device is present in the sensed evoked signal, the processing circuitry is configured to:
receive information of a sensed signal that is sensed in a continuous mode;
determine whether noise generated from delivery of the stimulation by the secondary medical device is present in the sensed signal that is sensed in the continuous mode; and
determine whether the noise generated from delivery of stimulation by the secondary medical device is present in the sensed evoked signal based on the determination of whether noise generated from delivery of the stimulation by the secondary medical device is present in the sensed signal that is sensed in the continuous mode.

7. The medical device of claim 1, wherein to process the sensed evoked signal based on a determination that noise generated from delivery of stimulation by the secondary medical device is present in the sensed evoked signal, the processing circuitry is configured to:
determine locations within the sensed evoked signal that corresponds to the noise generated from delivery of stimulation by the secondary medical device; and
output the sensed evoked signal for closed-loop stimulation control except of samples of the sensed evoked signal that correspond to the determined locations.

8. The medical device of claim 1, wherein to process the sensed evoked signal based on a determination that noise generated from delivery of stimulation by the secondary medical device is present in the sensed evoked signal, the processing circuitry is configured to:
determine locations within the sensed evoked signal that correspond to the noise generated from delivery of stimulation by the secondary medical device;
determine an estimate of the noise generated from delivery of stimulation by the secondary medical device; and
subtract the estimate of the noise from samples of the sensed evoked signal that correspond to the determined locations.

9. The medical device of claim 1, wherein to process the sensed evoked signal based on the determination of whether the noise generated from delivery of stimulation by the secondary medical device is present in the sensed evoked signal, the processing circuitry is configured to determine a rate at which to adjust stimulation parameters.

10. The medical device of claim 1, wherein the sensed evoked signal comprises a sensed evoked compound action potential (ECAP) that is sensed along a spinal cord of a patient.

11. A method for noise detection, the method comprising:
receiving information for a sensed evoked signal that is evoked due to stimulation delivery;
determining whether noise generated from delivery of stimulation by a secondary medical device is present in the sensed evoked signal; and
processing the sensed evoked signal based on the determination of whether the noise generated from delivery of stimulation by the secondary medical device is present in the sensed evoked signal.

12. The method of claim 11, wherein determining whether the noise generated from delivery of stimulation by the secondary medical device is present in the sensed evoked signal comprises executing a trained model that receives as input the information for the sensed evoked signal, wherein the trained model is trained using training data that includes sensed signals where noise generate from another device is present in the sensed signals.

13. The method of claim 11, wherein determining whether the noise generated from delivery of stimulation by the secondary medical device is present in the sensed evoked signal comprises:
accessing a template, the template representing a sensed signal where noise generated from delivery of stimulation is present in the sensed signal;
comparing the template to the sensed evoked signal; and
determining whether noise generated from delivery of stimulation by a secondary medical device is present in the sensed evoked signal based on the comparison.

14. The method of claim 11, wherein determining whether the noise generated from delivery of stimulation by the secondary medical device is present in the sensed evoked signal comprises:
determining a plurality of amplitude spikes in one or more instances of previously sensed evoked signals, an amplitude spike comprising a relatively narrow portion of the one or more instances of the previously evoked signals where an amplitude of the one or more previously evoked signals is greater than a threshold;
determining whether the plurality of amplitude spikes are periodic; and
determining whether the noise generated from delivery of stimulation by the secondary medical device is present in the sensed evoked signal based on whether the plurality of amplitude spikes are periodic.

15. The method of claim 11, wherein the sensed evoked signal comprises a first sensed evoked signal sensed using a first sensing channel, wherein determining whether the noise generated from delivery of stimulation by the secondary medical device is present in the sensed evoked signal comprises:
receiving information for a second sensed evoked signal that is sensed using a second sensing channel;
determining a similarity value indicative of a similarity between the first sensed evoked signal and the second sensed evoked signal; and
determining whether the noise generated from delivery of stimulation by the secondary medical device is present in the sensed evoked signal based on the similarity value.

16. The method of claim 11, wherein determining whether the noise generated from delivery of stimulation by the secondary medical device is present in the sensed evoked signal comprises:
receiving information of a sensed signal that is sensed in a continuous mode;
determining whether noise generated from delivery of the stimulation by the secondary medical device is present in the sensed signal that is sensed in the continuous mode; and
determining whether the noise generated from delivery of stimulation by the secondary medical device is present in the sensed evoked signal based on the determination of whether noise generated from delivery of the stimulation by the secondary medical device is present in the sensed signal that is sensed in the continuous mode.

17. The method of claim 11, wherein processing the sensed evoked signal based on a determination that noise generated from delivery of stimulation by the secondary medical device is present in the sensed evoked signal comprises:
determining locations within the sensed evoked signal that corresponds to the noise generated from delivery of stimulation by the secondary medical device; and
outputting the sensed evoked signal for closed-loop stimulation control except of samples of the sensed evoked signal that correspond to the determined locations.

18. The method of claim 11, wherein processing the sensed evoked signal based on a determination that noise generated from delivery of stimulation by the secondary medical device is present in the sensed evoked signal comprises:
determining locations within the sensed evoked signal that correspond to the noise generated from delivery of stimulation by the secondary medical device;
determining an estimate of the noise generated from delivery of stimulation by the secondary medical device; and
subtracting the estimate of the noise from samples of the sensed evoked signal that correspond to the determined locations.

19. The method of claim 11, wherein processing the sensed evoked signal based on a determination of whether the noise generated from delivery of stimulation by the secondary medical device is present in the sensed evoked signal comprises determining a rate at which to adjust stimulation parameters.

20. A computer-readable storage medium storing instructions thereon that when executed cause one or more processors to:
receive information for a sensed evoked signal that is evoked due to stimulation delivery;
determine whether noise generated from delivery of stimulation by a secondary medical device is present in the sensed evoked signal; and
process the sensed evoked signal based on the determination of whether the noise generated from delivery of stimulation by the secondary medical device is present in the sensed evoked signal.
